(19) United States
(12) Patent Application Publication
(10) Pub. No.: US 2011/0207194 A1
(43) Pub. Date: Aug. 25, 2011

(54) CLOSTRIDIUM SARTAGOFORMUM FOR THE GENERATION OF BIOGAS

(76) Inventors: Monika Reuter, Altotting (DE); Vera Duchow, Laupheim (DE); Daniel Vater, Bad Muskau (DE)

(21) Appl. No.: 12/735,374

(22) PCT Filed: Dec. 8, 2008
(86) PCT No.: PCT/DE2008/075014

§ 371 Date: Sep. 16, 2010

(30) Foreign Application Priority Data
Jan. 10, 2008 (DE) 10 2008 003 805.9

(51) Int. Cl. C12P 1/04; C12N 1/20

(52) U.S. Cl. 435/170; 435/252.7

(57) ABSTRACT

A process is described for the generation of biogas from biomass in a fermentation reactor, wherein a microorganism of the species Clostridium sartagoformum is added to the biomass.

### TECHNICAL FIELD

The invention relates to a process for the generation of biogas from biomass with the use of a microorganism of the species Clostridium sartagoformum.

### STATE OF THE TECHNOLOGY

Biogas plants generate methane through a process of microbial degradation of organic substances. In this, the biogas is formed in a multistage process of fermentation or rotting through the activity of anaerobic microorganisms, i.e. with exclusion of air.

From the chemical point of view, the organic material used as the substrate has a macromolecular structure which is degraded into lower molecular weight building blocks through metabolic activity of the microorganisms in the individual process steps of a biogas plant. However, the populations of microorganisms active in the fermentation of the organic fermentation substrate had previously only been inadequately characterized.

According to the current state of knowledge, four individual biochemical processes, running consecutively but also in parallel and interacting with one another, can be distinguished, which enable the degradation of organic fermentation substrates to the end products methane and carbon dioxide: hydrolysis, acidogenesis, acetogenesis and methanogenesis.

During hydrolysis, macromolecular organic compounds, often present in particulate form, are converted into soluble cleavage products by exoenzymes (e.g. cellulases, amylases, proteases and lipases) of fermentative bacteria. In the process, for example fats are broken down into fatty acids, carbohydrates, such as for example polysaccharides, into oligo- and monosaccharides and proteins into oligopeptides or amino acids. The gaseous products formed along with these predominantly consist of carbon dioxide.

During the acidogenesis, facultative and obligate anaerobic living bacteria, often identical with the hydrolyzing bacteria, metabolize the hydrolysis products (e.g. mono- and disaccharides, di- and oligopeptides, amino acids, glycerin, long-chain fatty acids) intracellularly to short-chain fatty or carboxylic acids, such as for example butyric, propionic and acetic acid, to short-chain alcohols such as for example ethanol and to the gaseous products hydrogen and carbon dioxide.

During the acetogenesis that follows, the short-chain fatty and carboxylic acids and the short-chain alcohols formed in the acidogenesis are taken up by acetogenic bacteria and after β-oxidation excreted again as acetic acid. Side-products of acetogenesis are CO₂ and molecular hydrogen (H₂).

The products of acetogenesis such as acetic acid, but also other substrates such as methanol and formate are converted to methane and CO₂ by methane-forming organisms during the methanogenesis, which proceeds obligate anaerobically. The CO₂ formed here and also the CO₂ formed during the other process steps, such as for example the hydrolysis, can in turn also be converted to methane by microorganisms with the H₂ that has formed.

As with any chemical conversion, increasing the yield of end products from a given quantity of educts is also a priority target in operating the process in the case of the production of biogas. For the generation of biogas from biomass, this means that as great as possible a quantity of methane should be formed from a given quantity of organic fermentation substrate.

At the same time, as high as possible a volume loading of the fermenter should be achieved. Volume loading of a fermenter is understood to mean the quantity of substrate fed into the fermenter, which is stated in kilograms of organic dry substance per cubic meter of fermenter volume per day. The quantity of biogas generated is strongly dependent on the volume loading of the fermenter, an increasingly large quantity of biogas being generated with increasing volume loading. A high volume loading thus makes the process of biogas generation increasingly economically profitable, but on the other hand results in increasing destabilization of the biological processes of the fermentation.

There is thus a further need for processes for the generation of biogas that enable an increased volume loading, as compared with the state of the art.

### DESCRIPTION OF THE INVENTION

The purpose of the invention is to provide a process for the generation of biogas that enables improved volume loading of the fermentation reactor, as compared with the state of the art.

This problem is solved according to the invention by the process for the generation of biogas as claimed in claim 1. Further advantageous details, aspects and embodiments of the present invention follow from the dependent claims, the description and the examples.

The present invention provides a process for the generation of biogas from biomass in a fermentation reactor. A microorganism of the species Clostridium sartagoformum is added to the biomass.

Surprisingly, it has been found that, through the addition of microorganisms of the species Clostridium sartagoformum to the fermentation substrate, both the volume loading of the fermenter can be increased and also the quantity of biogas formed is markedly increased. As shown in experimental studies, the addition of a microorganism of the species Clostridium sartagoformum effects an increase in the volume loading of a fermenter by more than 50%, without instability of the fermentation process arising. In parallel to the increased volume loading, the quantity of biogas formed is more than doubled. In addition, the specific yield of biogas increases, since markedly more of the organic dry substance is degraded than with no addition of micro-organisms of the species Clostridium sartagoformum. Through the increased degree of degradation, a markedly increased specific gas yield can be achieved with improved substrate utilization. Through the addition of microorganisms of the species Clostridium sartagoformum, the residence time of the fermentation substrate can be markedly shortened at constant gas yield, due to the increase in volume loading. The use of microorganisms of the species Clostridium sartagoformum therefore leads to a dramatic improvement in the efficiency and performance of biogas plants.

Fermentation in the sense of the present invention includes both anaerobic and also aerobic substance conversions through the action of microorganisms which lead to the generation of biogas. This explanation of the term “Fermentation” is stated under the heading “fermentation” on page 1306 of the Römpp Chemical Dictionary in the 9^{th}, expanded edition, published by Georg Thieme Verlag, which is incorporated herein by reference in its entirety.

The specific yield of biogas generated is calculated from the quantity of biogas generated divided by the quantity of organic dry substance. The question as to whether the generation of biogas in a certain fermenter under certain conditions proceeds in a satisfactory manner cannot be assessed solely on the basis of the quantity of biogas generated. Obviously, the quantity of biogas generated depends strongly on the quantity of substrate introduced, i.e. on the quantity of organic dry substance, which is stated in kilograms of organic dry substance. If instabilities in the fermentation process arise, then the specific gas yield decreases. With addition of microorganisms of the species Clostridium sartagoformum according to the invention, the volume loading can even be increased above the usual level, as a result of which a markedly increased quantity of gas is generated. Through the increased degree of degradation, a markedly increased specific gas yield can be achieved with better substrate utilization.

In the context of the present invention, the term “species of microorganisms” is understood to mean the relevant fundamental category of biological taxonomy. Species of microorganisms are identified and distinguished on the basis of their DNA sequences. Here not only microorganisms with a quite definite DNA sequence, but also genetic variants thereof up to a certain degree, fall under a particular species. To the appropriate person skilled in the art, it is known which strains of microorganisms fall within the term “species Clostridium sartagoformum”. From the state of the art, the isolation of microorganisms of the species Clostridium sartagoformum, for example from the feces of infants, is known (Stark, P. L.; Lee, A.; J. Pediatr. 1982; 100(3), p. 362-365). Microorganisms of the species Clostridium sartagoformum are not known in connection with, the production of biogas by fermentation of organic substrates,.

According to a preferred embodiment of the present invention, a microorganism of the species Clostridium sartagoformum is added in the form of a culture of microorganisms which predominantly consists of a microorganism of the species Clostridium sartagoformum. In fermentation substrates of biogas plants, microorganisms of the species Clostridium sartagoformum could be detected only in the smallest traces of less than 10⁻⁴% proportion of the total count of microorganisms present. Since the quantity of microorganisms isolated from their natural occurrence is insufficient for the addition of the organisms, propagation in the form of a culture is usually effected. In practice, it is found that the addition of the microorganisms to the fermentation substrate of a fermenter is most simply effected directly in the form of a culture of microorganisms.

The addition of the culture of Clostridium sartagoformum can be effected in the form of a culture suspension, in the form of dry, freeze-dried or moist cell pellets or also in the form of spore suspensions, spore preparations or dry, freeze-dried or moist spore pellets.

Since the various positive effects on the fermentation process already mentioned are connected with the microorganism species Clostridium sartagoformum, this species of microorganism should be present in the added culture in a quantity exceeding its natural occurrence. Of course, mixed cultures of any composition can be used for the addition. The only prerequisite is that the species Clostridium sartagoformum is present in a quantity enriched compared to its natural occurrence.

The determination of the contents of different species of microorganisms in mixed cultures presents no problem to the person skilled in the art. Thus, for example by means of fluorescent-labeled oligoprobes, the content of micro-organisms of the species Clostridium sartagoformum in a mixture can be specifically identified. As already mentioned, microorganisms of the species Clostridium sartagoformum are preferably added to the fermentation substrate in the form of cultures of microorganisms, the cultures of microorganisms consisting predominantly of microorganisms of the species Clostridium sartagoformum. If, in addition to the determination of the number of microorganisms of the species Clostridium sartagoformum the total number of microorganisms is also determined, then the content of microorganisms of the species Clostridium sartagoformum in the culture can be stated in percent. Microorganisms of the species Clostridium sartagoformum are the predominantly present species of microorganism in a mixed culture when they have the highest percentage content of the various species of microorganism present in the mixed culture.

According to preferred embodiments of the present invention the microorganism Clostridium sartagoformum makes up at least 10⁻⁴% of the total number of microorganisms present in the culture added to the fermentation substrate. Particularly preferably, the microorganism Clostridium sartagoformum makes up at least 10⁻²% of the total number of microorganisms present in the culture and especially preferably the microorganism Clostridium sartagoformum makes up at least 1% of the total number of microorganisms present in the culture.

According to further preferred embodiments, the microorganism Clostridium sartagoformum makes up at least 10% of the total number of microorganisms present in the culture, particularly preferably the microorganism Clostridium sartagoformum makes up at least 50% of the total number of microorganisms present in the culture and especially preferably the microorganism Clostridium sartagoformum makes up at least 90% of the total number of microorganisms present in the culture.

According to a quite particularly preferred embodiment, a pure culture of a microorganism of the species Clostridium sartagoformum is added. The pure culture of a microorganism comprises the progeny of one single cell which is isolated by a multistage process from a mixture of various micro-organisms. This multistage mechanism begins with the separation of a single cell from a cell population and requires that the colony arising from the cell through growth and cell division also remains separate. Through careful separation of a colony, renewed suspension in fluid and repeated plating out, pure cultures of microorganisms can be specifically obtained.

The isolation of a pure culture can, however, also be effected in liquid nutrient media, provided that the desired organism is numerically predominant in the starting material. By serial dilution of the suspension in the nutrient solution, it can be achieved that in the final dilution step only one cell is present. This cell then represents the basis for a pure culture. This explanation of the term “pure culture” and possible processes for creating this pure culture are stated under the heading “pure culture” on page 205 of the work “General Microbiology” by Hans G. Schlegel in the 7^{th} revised edition of 1992, published by Georg Thieme Verlag, which is incorporated herein by reference in its entirety.

The pure culture thus obtained is moreover also characterized biochemically by specific metabolic processes and activities, and by specific growth conditions. On the basis of the specific metabolic processes and activities, the addition of a pure culture of a fermentative microorganism can contribute to improved control of the complex biogas generation process to a remarkable extent.

According to a further preferred embodiment of the present invention, a microorganism of the species Clostridium sartagoformum is added as a component of at least one immobilized culture of microorganisms. Since the quantity of microorganisms isolated from their natural occurrence is not sufficient for the addition of the microorganisms, propagation in the form of culturing is usually performed. In practice, it is found that the addition of the microorganisms to the fermentation substrate of a fermenter is most simply effected in the form of an immobilized culture of microorganisms.

Since the various positive effects on the fermentation already mentioned are connected with the microorganism species Clostridium sartagoformum, this species of micro-organism should be present in the added immobilized culture in a quantity enriched compared to its natural occurrence. Of course, immobilized mixed cultures of any composition can be used for the addition. The only prerequisite is that microorganisms of the species Clostridium sartagoformum are contained in a quantity which exceeds their natural occurrence.

According to preferred embodiments of the present invention, the microorganism of the species Clostridium sartagoformum makes up at least 10⁻⁴% of the total number of microorganisms present in the immobilized culture added to the fermentation substrate. Particularly preferably, the microorganism of the species Clostridium sartagoformum makes up at least 10⁻²% of the total number of microorganisms present in the immobilized culture and especially preferably the microorganism of the species Clostridium sartagoformum makes up at least 1% of the total number of microorganisms present in the immobilized culture.

According to further preferred embodiments, the micro-organism of the species Clostridium sartagoformum makes up at least 10% of the total number of microorganisms present in the immobilized culture, particularly preferably the microorganism of the species Clostridium sartagoformum makes up at least 50% of the total number of microorganisms present in the immobilized culture and especially preferably the microorganism of the species Clostridium sartagoformum makes up at least 90% of the total number of microorganisms present in the immobilized culture.

According to a quite particularly preferred embodiment, at least one immobilized pure culture of a microorganism of the species Clostridium sartagoformum is added.

As support materials on which the microorganism Clostridium sartagoformum is immobilized, natural or synthetic polymers can be used. Preferably, gel-forming polymers are used. These have the advantage that bacteria can be taken up or deposited within the gel structure. Preferably, materials are used which slowly dissolve or are degraded in water, so that the release of the microorganism Clostridium sartagoformum takes place over a prolonged period.

Examples of suitable polymers are polyanilline, polypyrrole, polyvinylpyrolidone, polystyrene, polyvinyl chloride, polyvinyl alcohol, polyethylene, polypropylene, epoxy resins, polyethylenimines, polysaccharides such as agarose, alginate or cellulose, ethylcellulose, methyl-cellulose, carboxymethylethylcellulose, cellulose acetates, alkali cellulose sulfate, copolymers of polystyrene and maleic anhydride, copolymers of styrene and methyl methacrylate, polystyrene sulfonate, polyacrylates and polymethacrylates, polycarbonates, polyesters, silicones, cellulose phthalate, proteins such as gelatin, gum arabic, albumin or fibrinogen, mixtures of gelatin and waterglass, gelatin and polyphosphate or gelatin and copolymers of maleic anhydride and methyl vinyl ether, cellulose acetate butyrate, chitosan, polydialkyldimethylammonium chloride or mixtures of polyacrylic acids and polydiallyldimethyl-ammonium chloride and mixtures thereof. The polymeric material can also be crosslinked by means of normal crosslinkers such as glutaraldehyde, urea/formaldehyde resins or tanin compounds.

Alginates are found to be particularly advantageous as immobilizates since firstly they have no adverse effect on the activity of the microorganism Clostridium sartagoformum and secondly they are slowly degraded by microorganisms. Through the slow degradation of the alginate immobilizates, the enclosed microorganisms of the species Clostridium sartagoformum are gradually released.

For the immobilization, the microorganisms are mixed with a polymer gel and then cured in a suitable curing solution. For this, they are first mixed with a gel solution and then dripped from a suitable height into a curing solution. The exact procedures for immobilization are known to the person skilled in the art.

According to a further embodiment, close to the time of the addition of the microorganism of the species Clostridium sartagoformum, additional biomass is added to the fermentation reactor. Here “close to the time of relates to the time span which elapses between an addition of new substrate following the addition of the microorganisms. This time span should be kept as short as possible, hence the term “close to the time of also includes a parallel addition of the microorganism Clostridium sartagoformum and the new substrate.

Said time span can, however, also extend from several hours up to one or more days. Thus, by continuous addition of new substrate, the volume loading in the fermentation reactor can be continuously increased or held approximately constant, whereby the fermentation can be carried out at all volume loadings, preferably at a volume loading of ≧0.5 kg organic dry substance per m³ per day [kg oDS/m³d], more preferably at a volume loading of ≧4.0 kg oDS/m³d and particularly preferably at a volume loading of ≧8.0 kg oDS/m³d, which in comparison to the present state of the art corresponds to a more than twofold increase in the volume loading.

Hence, according to a further preferred embodiment, the volume loading in the fermentation reactor is continuously increased by continuous addition of biomass. Particularly preferably, the generation of biogas from biomass takes place at a volume loading of ≧0.5 kg oDS/m³d, especially preferably ≧4.0 kg oDS/m³d and quite particularly preferably ≧8.0 kg oDS/m³d.

The fermentation substrate used can in particular also have a high content of solid components. Through the addition of a hydrolytically active, fermentative microorganism of the species Clostridium sartagoformum these solid components are at least partially liquefied. Through the liquefaction of the fermentation substrate achieved on the basis of the addition of the microorganism Clostridium sartagoformum, thickening of the fermenter material can be avoided and specifically counteracted. A further liquid input into the fermentation substrate, in the form of water or liquid manure, during the fermentation can be avoided. Thus, there is a further advantage in the saving of the resource fresh water. Also advantageous is the preservation of the stirrability and pumpability of the substrate thus attained. As a result, stirrers and pumps are protected and markedly less energy is necessary for the stirring operation.

According to a further embodiment, the generation of biogas from biomass takes place with constant thorough mixing of the fermentation substrate. Through the constant thorough mixing of the fermentation substrate, the cultures of Clostridium sartagoformum can be better distributed in the fermentation substrate. In addition, the biogas formed can be better drawn off from the fermentation process.

In addition, the constant thorough mixing of the fermentation substrate results in a uniform heat distribution in the fermentation reactor. Measurements of the temperature in the fermentation reactor, which were made at periodic intervals, but also continuously, showed that the fermentation substrate is efficiently fermented in a temperature range from 20° C. to 80° C., preferably at about 40° C. to 50° C. These temperature ranges are therefore preferable in the context of the present invention. As well as the hydrolysis, in particular the last stage of the fermentation process, namely the formation of methane by methanogenic microorganisms, takes place particularly efficiently at elevated temperatures.

Hence, the generation of biogas from biomass preferably takes place at a temperature of 20° C. to 80° C. and particularly preferably at a temperature of 40° C. to 50° C.

All the embodiments of the present invention are of course not restricted to single-stage processes for the production of biogas. The use of microorganisms of the species Clostridium sartagoformum can also take place in two- or multistage processes.

According to a further embodiment, a fermentation substrate and a microorganism of the species Clostridium sartagoformum are added continuously. The continuous operation of a fermentation reactor with a stable microbial biocenosis should lead to continuous production of biogas, whereby the interruption of substrate feed to the fermentation because of a process malfunction should be decreased.

Likewise, the implementation of this fermentation process and the processes connected therewith in discontinuous operation, for example “batch” fermentation, is also conceivable. Thus according to a further embodiment the microorganism of the species Clostridium sartagoformum can for example be added to the fermentation substrate at regular intervals during the fermentation. The addition of the microorganism of the species Clostridium sartagoformum at regular intervals leads to an increase in the live cell count and hence to an improved course of the fermentative process, for example the hydrolysis, with simultaneously improved utilization of the fermentation substrate for the fermentation.

According to a preferred embodiment of the present invention, the microorganism of the species Clostridium sartagoformum is added to the fermentation substrate in a quantity such that after addition the content of the microorganism of the species Clostridium sartagoformum makes up between 10⁻⁸% and 50% of the total number of microorganisms present in the fermentation substrate. In particular, depending on the fermenter size and hence depending on the quantity of fermentation substrate, an addition of very markedly different quantities of microorganisms can be necessary to achieve the desired effect.

Both the determination of the total number of microorganisms in the fermentation substrate and also the determination of the contents of different species of microorganisms in the fermentation substrate presents no problem to the person skilled in the art. Thus, for example by means of fluorescent-labeled oligoprobes, the content of microorganisms of various microorganisms in the fermentation substrate can be specifically identified.

Particularly preferably, a microorganism of the species Clostridium sartagoformum is added to the fermentation substrate in a quantity such that after addition the content of the microorganism of the species Clostridium sartagoformum makes up between 10⁻⁶% and 25% of the total number of microorganisms present in the fermentation substrate. Especially preferably, the microorganism of the species Clostridium sartagoformum is added to the fermentation substrate in a quantity such that, after addition, the content of the microorganism of the species Clostridium sartagoformum makes up between 10⁻⁴% and 10% of the total number of microorganisms present in the fermentation substrate. Quite particularly preferably, the microorganism of the species Clostridium sartagoformum is added to the fermentation substrate in a quantity such that, after addition, the content of the microorganism of the species Clostridium sartagoformum makes up between 10⁻³% and 1% of the total number of microorganisms present in the fermentation substrate.

It should once again be pointed out that the addition of microorganisms of the species Clostridium sartagoformum can take place at any time in the fermentation process, and in particular, microorganisms of the species Clostridium sartagoformum can also be used for inoculation of fermentation substrate during the first start-up or a renewed start-up of a fermenter.

Likewise it is possible to add microorganisms of the species Clostridium sartagoformum for stabilization of the fermentation during malfunctions of the fermentation process. Such malfunctions can be recognized in good time by monitoring certain characteristic parameters of the fermentation. Characteristic parameters give information about the quality of an operating fermentation process for the production of biogas. Such characteristic parameters are not only the quantity of biogas generated and the methane content of the biogas generated but also, for example, the hydrogen content of the biogas generated, the pH of the fermentation substrate, the redox potential of the fermentation substrate, the carboxylic acid content of the fermentation substrate, the contents of various carboxylic acids in the fermentation substrate, the hydrogen content of the fermentation substrate, the content of dry substance in the fermentation substrate, the content of the organic dry substance in the fermentation substrate, the viscosity of the fermentation substrate and the volume loading of the fermentation reactor.

The present invention also includes the use of a microorganism of the species Clostridium sartagoformum for the fermentative generation of biogas from biomass.

Likewise, the present invention includes the strain of the microorganism Clostridium sartagoformum SBG1, as deposited under the No. DSM 19861. The microorganism Clostridium sartagoformum SBG1 was deposited as a pure culture at the German Collection of Microorganisms and Cell Cultures GmbH in Braunschweig in accordance with the Budapest Treaty. The designation reads: Clostridium sartagoformum SBG1 with the deposition number DSM 19861.

Bacteria of the species Clostridium sartagoformum can be isolated from the fermentation substrate of a fermenter by methods known to the person skilled in the art. During this, a suitable substrate from a fermenter is introduced into a selection medium and cultured over a prolonged period until finally individual colonies of microorganisms are isolated from the selection medium. After amplification of the microbial DNA obtained therefrom by PCR, microorganisms of the species Clostridium sartagoformum can be selected on the basis of the 16S rRNA.

Bacteria Clostridium sartagoformum SBG1 were isolated from the fermentation substrate of a secondary fermenter. For this, nitrogen and carbon dioxide were passed through a liquid selection medium, then Na₂S was added to the selection medium and autoclaved (20 mins at 121° C.). Then, the biomass obtained from the secondary fermenter was introduced into the selection medium and cultured for at least one week at a temperature of at least 30° C. A sample taken from the liquid selection medium was applied onto a solid selection medium and then the colonies of microorganisms which had grown on the solid selection medium were selected. After amplification of the microbial DNA obtained by PCR, a comparison with known DNA sequences was performed.

After the bacteria Clostridium sartagoformum SBG1 had been successfully isolated from the fermentation substrate of the secondary fermenter, these microorganisms were subjected to sequence analysis. The 16S rRNA sequence SEQ ID No. 1 comprises 948 nucleotides. As the closest relative, a non-cultured bacterial clone aaa62b07 with the gene sequence SEQ ID No. 2 was identified. A comparison of the sequences shows that a total of 9 exchanges of nucleotides or gaps are present. With a Clostridium sartagoformum SBG1 sequence length of 948 nucleotides, an identity of more than 99.2% is calculated by means of the FASTA algorithm.

The present invention also includes microorganisms with a nucleic acid which has a nucleotide sequence which contains a sequence region which has more than 99.2% sequence identity with the nucleotide sequence SEQ ID No. 1. Particularly preferably, the nucleotide sequence contains a sequence region which has more than 99.4% sequence identity with the nucleotide sequence SEQ ID No. 1 and especially preferably the nucleotide sequence contains a sequence region which has more than 99.6% sequence identity with the nucleotide sequence SEQ ID No. 1.

According to further preferred embodiments, the microorganism has a nucleotide sequence which contains a sequence region which has more than 99.8% sequence identity with the nucleotide sequence SEQ ID No. 1 and particularly preferably the nucleotide sequence contains a sequence region which has more than 99.9% sequence identity with the nucleotide sequence SEQ ID No. 1.

According to a quite particularly preferred embodiment, the nucleotide sequence contains a sequence region which corresponds to the nucleotide sequence SEQ ID No. 1.

The present invention thus also includes all micro-organisms with a nucleic acid whose nucleotide sequence has at least one sequence region which, compared to the nucleotide sequence SEQ ID No. 1, has a nucleotide exchange at only one position. Also included are all microorganisms, whose DNA sequence has at least one sequence region which, compared to the nucleotide sequence SEQ ID No. 1, has a nucleotide exchange at only two positions. In -addition, all microorganisms are included whose DNA sequence has at least one sequence region which, compared to the nucleotide sequence SEQ ID No. 1, has a nucleotide exchange at only three positions.

In addition, the present invention also includes all microorganisms whose DNA sequence has at least one sequence region which, compared to the nucleotide sequence SEQ ID No. 1, has nucleotide exchanges at four positions. Also included are all microorganisms whose DNA sequence has at least one sequence region which, compared to the nucleotide sequence SEQ ID No. 1, has nucleotide exchanges at five positions. As well as this, all microorganisms whose DNA sequence has at least one sequence region which, compared to the nucleotide sequence SEQ ID No. 1, has nucleotide exchanges at six positions are included.

Apart from this, the present invention also includes all microorganisms whose DNA sequence has at least one sequence region which, compared to the nucleotide sequence SEQ ID No. 1, has nucleotide exchanges at seven positions. Also included are all microorganisms whose DNA sequence has at least one sequence region which, compared to the nucleotide sequence SEQ ID No. 1, has nucleotide positions at eight positions.

It goes without saying that the exchanges of the nucleotides can be present at any position of the DNA sequence. In particular, the exchanges can be present at positions at any distance from one another. If, for example, in comparison to the nucleotide sequence SEQ ID No. 1, six nucleotides are exchanged, then these six exchanged nucleotides can be present adjacent to one another. In this case, a six nucleotide long fragment of the nucleotide sequence SEQ ID No. 1 would be changed. Likewise, however, the six exchanged nucleotides could, for example, each be 100 nucleotides away from one another. Thus, the exchanges can be present in any combinations.

Likewise, said exchanges can also be present in the form of gaps. The present invention thus also includes all micro-organisms with a nucleic acid whose nucleotide sequence has at least one sequence region in which, compared to the nucleotide sequence SEQ ID No. 1, a nucleotide is missing at only one position. Also included are all microorganisms whose DNA sequence has at least one sequence region in which, compared to the nucleotide sequence SEQ ID No. 1, nucleotides are missing at only two positions. Apart from this, all microorganisms whose DNA sequence has at least one sequence region in which, compared to the nucleotide sequence SEQ ID No. 1, nucleotides are missing at only three positions are included.

In addition, the present invention also includes all microorganisms whose DNA sequence has at least one sequence region in which, compared to the nucleotide sequence SEQ ID No. 1, nucleotides are missing at four positions. Also included are all microorganisms whose DNA sequence has at least one sequence region in which in comparison to the nucleotide sequence SEQ ID No. 1 nucleotides are missing at five positions. Apart from this, all microorganisms whose DNA sequence has at least one sequence region in which, compared to the nucleotide sequence SEQ ID No. 1, nucleotides are missing at six positions are included.

Apart from this, the present invention also includes all microorganisms whose DNA sequence has at least one sequence region in which, compared to the nucleotide sequence SEQ ID No. 1, nucleotides are missing at seven positions. Also included are all microorganisms whose DNA sequence has at least one sequence region in which, compared to the nucleotide sequence SEQ ID No. 1, nucleotides are missing at eight positions.

It goes without saying that the nucleotides can be missing at any position of the DNA sequence. In particular, the gaps can be present at positions at any distance from one another. If for example in comparison to the nucleotide sequence SEQ ID No. 1 six nucleotides are missing, then these six missing nucleotides can be present adjacent to one another in the nucleotide sequence SEQ ID No. 1. In this case, a six nucleotide long fragment of the nucleotide sequence SEQ ID No. 1 would be missing. Likewise, however, the six missing nucleotides could for example each lie 100 nucleotides away from one another in the nucleotide sequence SEQ ID No. 1. Thus the gaps can be present in any combinations.

The present invention also includes a culture of micro-organisms suitable for use in a process for the fermentative generation of biogas from biomass, wherein in the culture of microorganisms a microorganism Clostridium sartagoformum SBG1 as deposited under the No. DSM 19861 is present wherein the microorganism makes up at least 10⁻⁴% of the total number of microorganisms present in the culture.

The present invention also includes a culture of micro-organisms suitable for use in a process for the fermentative generation of biogas from biomass, wherein, in the culture of microorganisms, a microorganism which has a nucleotide sequence which contains a sequence region which has at least 99.2% sequence identity with the nucleotide sequence SEQ ID No. 1 is present and wherein the microorganism makes up at least 10⁻⁴% of the total number of microorganisms present in the culture.

Preferably, in the culture of microorganisms suitable for use in a process for the fermentative generation of biogas from biomass, a microorganism is present that has a nucleotide sequence with a sequence region having more than 99.4% sequence identity with the nucleotide sequence SEQ ID No. 1. Especially preferably, the nucleotide sequence contains a sequence region which has more than 99.6% sequence identity with the nucleotide sequence SEQ ID No. 1.

According to further preferred embodiments, in the culture of microorganisms suitable for use in a process for the fermentative generation of biogas from biomass, a microorganism which possesses a nucleotide sequence with a sequence region which has more than 99.8% sequence identity with the nucleotide sequence SEQ ID No. 1 is present. Quite particularly preferably the nucleotide sequence contains a sequence region which has more than 99.9% sequence identity with the nucleotide sequence SEQ ID No. 1.

According to a quite particularly preferred embodiment, in the culture of microorganisms suitable for use in a process for the fermentative generation of biogas from biomass, a microorganism which has a nucleotide sequence which contains a sequence region which corresponds to the nucleotide sequence SEQ ID No. 1 is present.

According to further preferred embodiments, the micro-organism Clostridium sartagoformum makes up at least 10⁻²%, preferably at least 1%, of the total number of microorganisms present in the culture. Particularly preferably the microorganism Clostridium sartagoformum makes up at least 10%, especially preferably at least 25% of the total number of microorganisms present in the culture.

Quite particularly preferably the microorganism Clostridium sartagoformum makes up at least 50%, in particular at least 75% of the total number of microorganisms present in the culture.

Likewise preferable are embodiments according to which the microorganism Clostridium sartagoformum makes up at least 90% of the total number of microorganisms present in the culture. Particularly preferably, it is a pure culture of microorganisms suitable for use in a process for the fermentative generation of biogas from biomass, it being a pure culture of the microorganism Clostridium sartagoformum SBG1 as deposited under the No. DSM 19861 or as characterized above in relation to its nucleotide sequence.

Particularly preferably, in the cases described above it is an immobilized culture of microorganisms.

The present invention also includes an immobilized culture of microorganisms suitable for use in a process for the fermentative generation of biogas from biomass, wherein in the immobilized culture of microorganisms a microorganism Clostridium sartagoformum as deposited under the No. DSM 19861 is present.

The present invention also includes an immobilized culture of microorganisms suitable for use in a process for the fermentative generation of biogas from biomass, wherein in the immobilized culture of microorganisms a microorganism which has a nucleotide sequence which contains a sequence region which has at least 99.2% sequence identity with the nucleotide sequence SEQ ID No. 1 is present.

Preferably, in the immobilized culture of microorganisms suitable for use in a process for the fermentative generation of biogas from biomass, a microorganism which possesses a nucleotide sequence with a sequence region which has more than 99.4% sequence identity with the nucleotide sequence SEQ ID No. 1 is present. Especially preferably the nucleotide sequence contains a sequence region which has more than 99.6% sequence identity with the nucleotide sequence SEQ ID No. 1.

According to further preferred embodiments, in the immobilized culture of microorganisms suitable for use in a process for the fermentative generation of biogas from biomass, a microorganism which has a nucleotide sequence which possesses a sequence region which has more than 99.8% sequence identity with the nucleotide sequence SEQ ID No. 1 is present. Quite particularly preferably, the nucleotide sequence contains a sequence region which has more than 99.9% sequence identity with the nucleotide sequence SEQ ID No. 1.

According to a quite particularly preferred embodiment, in the immobilized culture of microorganisms suitable for use in a process for the fermentative generation of biogas from biomass, a microorganism which has a nucleotide sequence which contains a sequence region which corresponds to the nucleotide sequence SEQ ID No. 1 is present.

### BRIEF DESCRIPTION OF THE DIAGRAMS

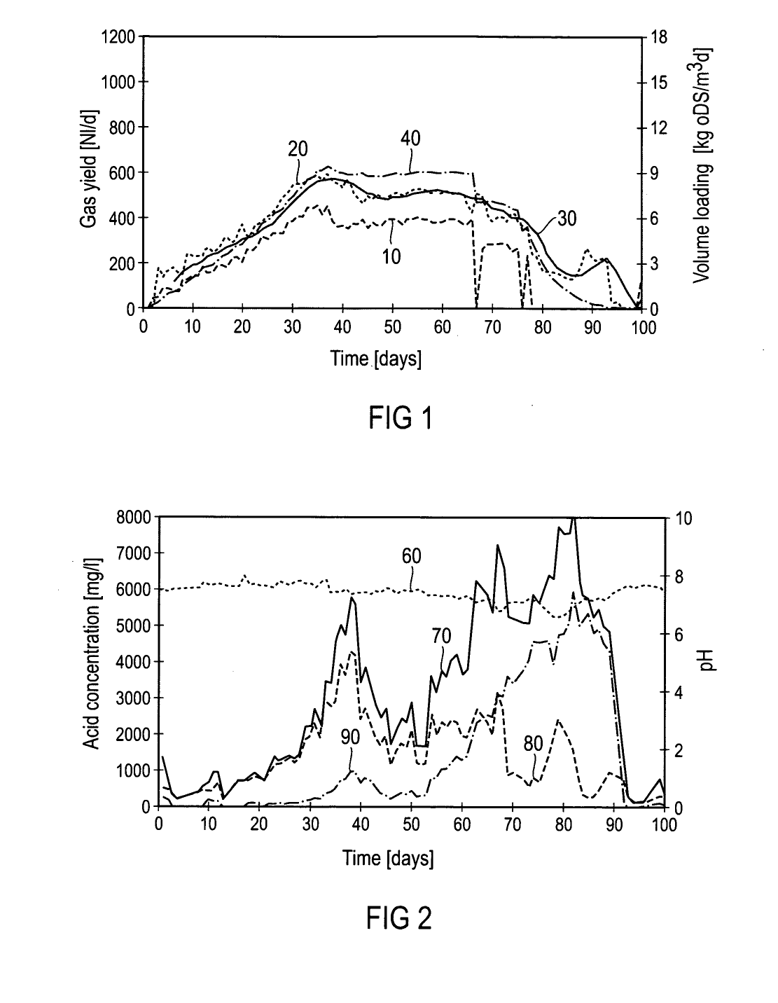

To illustrate the invention and explain its advantages, practical examples are given below. The practical examples will be explained in more detail in connection with FIGS. 1 to 6. It goes without saying that the statements made in connection with the practical examples are not intended to limit the invention. They show:

FIG. 1 Measurement results from a fermentation: the quantity of biogas generated, the 6-day moving average of the quantity of biogas generated, the theoretical gas production and the volume loading of the fermenter are plotted against time;

FIG. 2 Measurement results from the fermentation according to FIG. 1: the concentration of acetic acid, the concentration of propionic acid, the acetic acid equivalent and the pH are plotted against time;

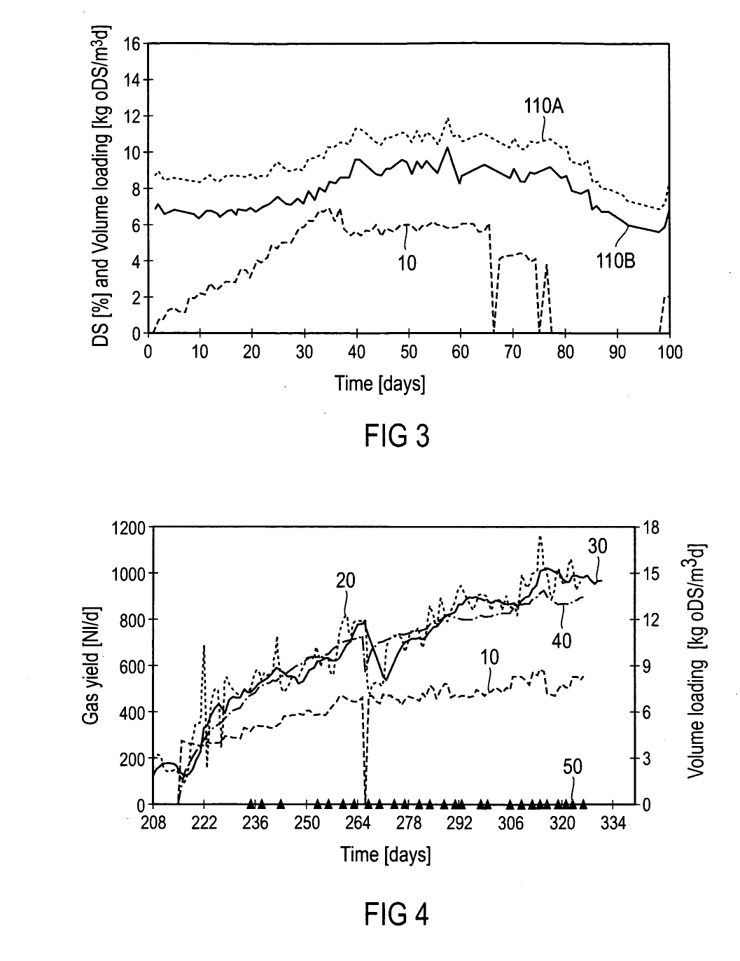

FIG. 3 Measurement results from the fermentation according to FIG. 1: the volume loading of the fermenter, the percentage dry substance content in the fermentation substrate and the percentage content of the organic dry substance are plotted against time;

FIG. 4 Measurement results from a fermentation with addition of Clostridium sartagoformum SBG1 and otherwise identical conditions to those in the fermentation according to FIGS. 1 to 3: the quantity of biogas generated, the 6-day moving average of the quantity of biogas generated, the theoretical gas production and the volume loading of the fermenter are plotted against time;

FIG. 5 Measurement results from the fermentation according to FIG. 4: the concentration of acetic acid, the concentration of propionic acid, the acetic acid equivalent and the pH are plotted against time;

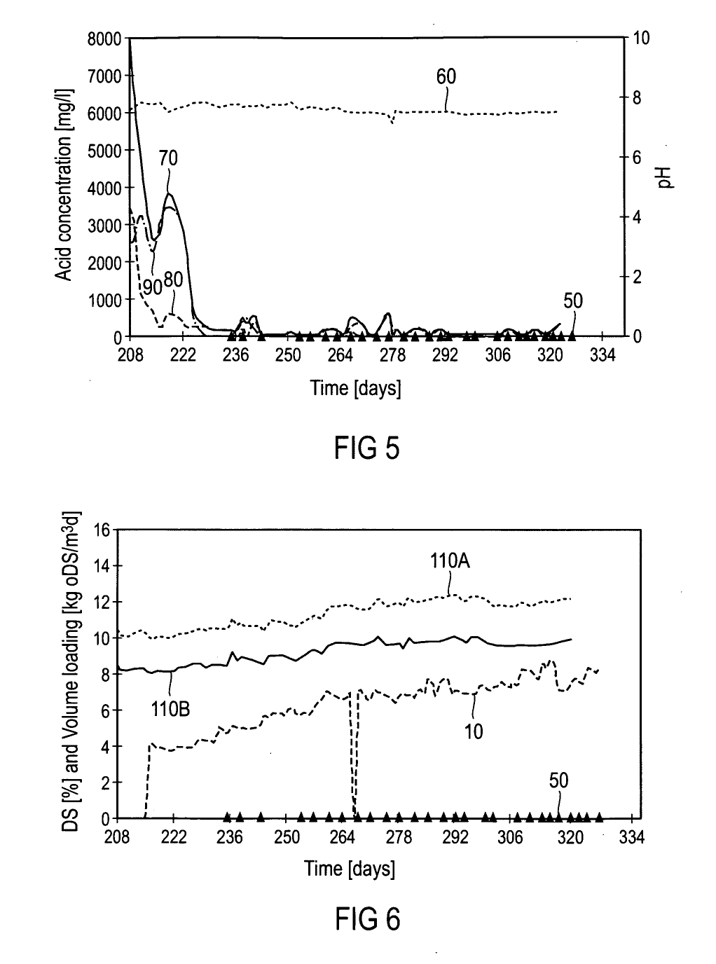

FIG. 6 Measurement results from the fermentation according to FIG. 4: the volume loading of the fermenter, the percentage dry substance content in the fermentation substrate and the percentage content of the organic dry substance are plotted against time.

### MODES OF IMPLEMENTATION OF THE INVENTION

### COMPARATIVE EXAMPLE

FIG. 1 shows measurement results for various characteristic parameters during a fermentation process in an experimental fermenter with a volume of 150 ; under realistic plant conditions. The curve marked with the reference symbol 10 shows the variation with time of the volume loading of the fermenter in kilograms of organic dry substance per cubic meter per day (kg oDS/m³d) and the curve marked with the reference symbol 20 the variation with time of the total biogas generated in standard liters (gas volume at 273.15 K and 1013 mbar) per day (NI/d). The variation with time of the 6-day moving average of the total biogas generated in [NI/d] is labeled with the reference symbol 30 and the variation with time of the theoretical gas production in [NI/d] is marked with the reference symbol 40.

The Association for Technology and Structures in Agriculture (KTBL) and also the State Institute for Agriculture have issued standard values which state roughly what quantities of biogas are to be expected in a stable fermentation depending on the substrate used. Thus, these theoretical standard values can reflect the quantity of biogas that can theoretically be generated. Alternatively, standard values issued by other institutes in other states can also be used. The variation with time in the theoretic gas production 40 in [NI/d] was calculated according to such standard values.

From FIG. 1 it can be seen that in the course of the first 35 days a continuous and stable fermentation process takes place, which is characterized by a rise in the volume loading to about 6.5 kg oDS/m³d and by formation of biogas continuously increasing with the volume loading. From day 36, a deviation in the quantity of biogas generated from the theoretical gas production can be observed. Because of an uncontrolled high acid concentration in the fermentation substrate, which is shown in FIG. 2, the volume loading is kept approximately constant from day 38 by interrupting the increase in the substrate input. Since however the acid concentrations remains uncontrollable, on day 67 and on day 76 no substrate is introduced and from day 78 the introduction of substrate is completely discontinued. It was found that a maximum volume loading of 5.5 kg oDS/m^{s}d could be attained. At a higher volume loading, stable operation is no longer possible on account of the breakdown of the fermentation process.

FIG. 2 shows the time-dependent development of the concentrations of characteristic carboxylic acids during the fermentation process already explained in connection with FIG. 1. The variation in the pH (curve marked with 60), the acetic acid equivalent for the determination of the volatile fatty acids (curve marked with 70), the acetic acid concentration (curve marked with 80) and the propionic acid concentration (curve marked with 90) with time are shown. In this, the acid concentrations are stated in milligrams per liter of fermentation substrate (mg/l).

The determination of volatile fatty acids as a summation parameter in the fermentation substrate was performed by steam distillation of a fermentation substrate sample acidified with phosphoric acid. The distillate was then titrated with sodium hydroxide solution against phenolphthalein. Alternatively or for differentiation of the individual carboxylic acids, a determination by gas chromatography is also possible.

It was found that the pH in the first 35 days of the experiment was almost stable at pH 7.5. Owing to the uncontrolled high acid concentration from day 36, as explained in connection with FIG. 1, the substrate input was firstly not increased further and completely discontinued on day 67, on day 76 and from day 78. Together with the uncontrolled high acid concentration, the pH falls slightly into a weakly acid range, with which an unstable fermentation process begins to emerge. The pH then only swings into the neutral to slightly alkaline range again when the acid concentration sinks towards zero. This recovery in the pH can be explained through the microbial degradation of the acids in the fermentation substrate, supported by the lack of further substrate input.

FIG. 3 shows the dry substance content of the fermenter contents as a function of the volume loading during the fermentation process already explained in connection with FIGS. 1 and 2. The variation of the volume loading in [kg oDS/m³d] (curve labeled with the reference symbol 10), the percentage dry substance content (curve labeled with the reference symbol 110A) and the percentage content of the organic dry substance (curve labeled with the reference symbol 110B) with time are shown. The percentage dry substance content gives the total mass of organic and inorganic substances such as, for example, sand.

From FIG. 3 it can be seen that, with the rise in the acid concentration on day 36 shown in FIG. 2, the percentage dry substance content and the percentage content of dry organic substance also rises. In spite of the reduction in the volume loading undertaken on day 38, which remains almost constant at 5.5 to 6.0 kg oDS/m³d between days 38 to 67, organic dry substance 110B, which can no longer be adequately fermented, builds up. The biological processes recover so that the organic dry substance is again fermented only with the discontinuation of the substrate input from day 38, as is clear from the decline in the content of the dry substance 110B.

Clostridium sartagoformum SBG1

Bacteria of the species Clostridium sartagoformum SBG1 were successfully isolated from the fermentation substrate of a secondary fermenter. The deposition of the organism as a pure culture was effected at the German Collection of Microorganisms and Cell Cultures GmbH in accordance with the Budapest Treaty (Clostridium sartagoformum SBG1 with the deposition number DSM 19861).

The isolation of the microorganisms was effected using a selection medium which contained carboxymethylcellulose as the only carbon source. Carboxymethylcellulose has a very great similarity to the cellulose contained in fermentation substrates of biogas plants and, in addition, owing to the linkage of the hydroxyl groups with carboxymethyl groups (—CH₂—COOH—), has improved solubility in aqueous medium. The medium used for the selection of Clostridium sartagoformum SBG1 was gassed with N₂ and CO₂ so that the selection could take place under anaerobic conditions. Contained residual oxygen was then reduced by means of 0.5 g/l Na₂S.

The selection medium was then inoculated with the supernatant from material from a secondary fermenter. After culturing for one week at 40° C., individual rods were seen on microscopic analysis. A further selection of the liquid cultures was effected by plating out onto anaerobic carboxymethylcellulose plates. The cell material of the colonies that grew was used for the amplification of the microbial DNA using the colony PCR method according to a standard program. After the sequence analysis of the colonies, phylogenetic comparison of the sequence data generated therefrom by means of the BLAST program (basic local alignment search tool) of the database www.ncbi.nlm.nih.gov showed that the sequence obtained can be assigned to the organism Clostridium sartagoformum as the closest relative.

Experiments with a pure culture of the hydrolytically active, fermentative microorganism Clostridium sartagoformum SBG1 showed that addition to the very slow-flowing, viscous carboxymethylcellulose-containing selection medium leads to a progressive liquefaction of the medium.

### EXAMPLE

FIG. 4 shows measurement results for various characteristic parameters during a fermentation process in an experimental plant under the plant conditions described in connection with FIGS. 1 to 3. The curve marked with the reference symbol 10 shows the variation of the volume loading of the fermenter in kilograms dry organic substance per cubic meter per day (kg oDS/m³d) with time and the curve labeled with the reference symbol 20 the variation in the total biogas generated in standard liters (gas volume at 273.15 K and 1013 mbar) per day (NI/d) with time. The variation in the 6-day moving average of the total biogas generated in [NI/d] with time is labeled with the reference symbol 30 and the variation of the theoretical gas production [NI/d] with time is marked with the reference symbol 40.

In contrast to the fermentation described in connection with FIGS. 1 to 3, in the fermentation described below with reference to FIGS. 4 to 6 the microorganism Clostridium sartagoformum was added according to the invention to the fermentation substrate. The times of addition of Clostridium sartagoformum SBG1 are shown by triangles on the x axis marked with the reference symbol 50.

In the practical example described, a pure culture of Clostridium sartagoformum was used. Likewise however, mixed cultures with a content of Clostridium sartagoformum can also be used.

From day 215, a continuous increase in the volume loading from ca. 4.25 kg oDS/m³d to ca. 8.5 kg oDS/m³d on day 314 was effected. Within this period, a pure culture of Clostridium sartagoformum SBG1 was added to the fermentation substrate several times. The fall in the volume loading on day 266 is attributable to a technical problem which on that day led to a breakdown in the substrate feed in the biogas plant.

The first use of a pure culture of Clostridium sartagoformum SBG1 took place on experiment day 235. At the time of the first addition of microorganisms, the fermenter was operated with a volume loading of about 4.5 kg organic dry substance/m³ per day. For this, the cell mass from 1 l of a preculture of Clostridium sartagoformum SBG1 which had been incubated for 5 days at a temperature of about 40° C. was used. The cell count of this preculture had a cell density of ca. 2.0×10⁸ cells/ml with a live cell content of over 90%. While the addition was at first performed twice weekly on the 1 l scale, from experiment day 277 the quantity added was doubled, so that an addition of the cell mass from 2 l of a preculture of Clostridium sartagoformum SBG1 was performed twice weekly.

From experiment day 293, cell mass from 2 l of a preculture of Clostridium sartagoformum SBG1 was added three times weekly. In addition, the incubation time of the preculture was extended to at least 7 days, as a result of which the cell density rose to 1×10⁹ cells/ml on average. Through the addition of the pure cultures of Clostridium sartagoformum SGB1, the volume loading in the further course of the experiment could be increased to a maximum value of about 8.5 kg oDS/m³d, reached on day 314.

In parallel to the increasing volume loading, an increase in the biogas yield could be observed. Here a correspondence between the quantity of biogas generated in standard liters/day [NI/d] and the theoretical gas production in standard liters/day [NI/d] could be observed, wherein from about day 288 the quantity of biogas generated remained almost constantly above the biogas yield theoretically to be expected. This is attributable to the increased addition of the Clostridium sartagoformum SBG1 pure culture from day 277.

In addition, after experiment days 298 and 308 it can be observed that the gas yield briefly rose markedly above the theoretical value, and subsequently again approximated to the theoretical curve. These increases in gas production are associated with an increase in the quantity of microorganisms added at those times. From this, it can be concluded that, after an adaptation phase in which a basic concentration of microorganisms has to be built up, an increase in the cell count leads to acceleration of the hydrolysis.

FIG. 5 shows the development of the concentrations of characteristic carboxylic acids as a function of time during the fermentation process already described in connection with FIG. 4. The variation with time of the pH (curve labeled with the reference symbol 60), the acetic acid equivalent for determination of the volatile fatty acids in [mg/l], marked with the reference symbol 70, the acetic acid concentration in [mg/l] (curve labeled with the reference symbol 80) and the propionic acid concentration in [mg/l] (curve labeled with the reference symbol 90) are shown.

From FIG. 5 it can be seen that the pH remained constant during the fermentation process and lay in the neutral to slightly alkaline range between pH 7 and 8. At the start of the fermentation process, a drastic fall in the concentration of the acetic acid equivalent, the acetic acid concentration and the propionic acid concentration could already be discerned. Overall, the fatty acid concentration remained persistently low, and no enrichment of longer-chain fatty acids could be observed.

Neither the drastically increased substrate input already described in connection with FIG. 4, nor the interrupted substrate input on day 266, caused prominent peaks in the concentration of the carboxylic acids. This means that the intermediate products generated in the hydrolysis and in the subsequent acido- and acetogenesis are converted to biogas during the methanogenesis as the last step of the biogas synthesis. Thus, it has been shown that the fermentation processes proceed well even with prolonged high loading, so that no enrichment in organic intermediate products takes place and consequently the long-term stability of the fermentation process is ensured.

FIG. 6 shows the dry substance content of the fermenter contents and the volume loading during the fermentation process already explained in connection with FIGS. 4 and 5. The variation in the volume loading in [kg oDS/m³d] (curve labeled with the reference symbol 10), the percentage dry substance content (curve labeled with the reference symbol 110A) and the percentage content of the organic dry substance (curve labeled with the reference symbol 110B) with time are shown.

At the time of the first addition of a pure culture of Clostridium sartagoformum SBG1 on experiment day 235, the fermenter was operated with a volume loading of about 4.5 kg organic dry substance per m³ per day. The volume loading of the plant could be further increased as a result of the addition of Clostridium sartagoformum SBG1. At the same time, the percentage content of dry substance or organic dry substance remained almost constant. This observation renders it obvious that during the fermentation of the fermentation substrate no buildup of unfermented organic dry substance occurs. The addition of pure cultures of Clostridium sartagoformum SBG1 thus contributes to a continuous conversion of the contained dry mass in the fermentation substrate, which in turn leads to continuous fermentation, while the build-up of dry substance is decreased.

In phases in which the biogas plant is operated at constant volume loading, a decrease in the dry substance can even be observed, which leads to the conclusion that the Clostridium sartagoformum SBG1 microorganisms with their hydrolytic metabolic activity not only decrease the buildup of dry substance in the fermenter, but also improve the hydrolytic conversion of this dry substance.

Thus FIGS. 4 to 6 confirm the positive effect of the addition of the hydrolytically active, fermentative microorganism Clostridium sartagoformum SBG1 on the hydrolysis of organic dry substance. Through the addition of microorganisms of the species Clostridium sartagoformum the volume loading of a fermenter under otherwise identical conditions can be increased from about 5.5 kg oDS/m³d to around 8.5 kg oDS/m³d, i.e. by more than 50%, without there being even a hint of instability of the fermentation process. In parallel to the increased volume loading, the quantity of biogas formed is more than doubled. In addition, the specific yield of biogas rises, since markedly more of the organic dry substance is degraded than without the addition of micro-organisms of the species Clostridium sartagoformum. The use of microorganisms of the species Clostridium sartagoformum results in a dramatic improvement in the efficiency and performance of biogas plants.

What is claimed is:

1-38. (canceled)

39. A process for the generation of biogas from biomass in a fermentation reactor, comprising the step of adding a micro-organism of the species Clostridium sartagoformum the biomass in a form of a culture of microorganisms, wherein the microorganism of the species Clostridium sartagoformum makes up at least 1% of the total number of microorganisms present in the culture.

40. The process as claimed in claim 39, wherein, in the culture of microorganisms, the microorganism of the species Clostridium sartagoformum makes up at least 10% of the total number of microorganisms present in the culture.

41. The process as claimed in claim 39, wherein a pure culture of the microorganism of the species Clostridium sartagoformum is added.

42. The process as claimed in claim 39, wherein the microorganism of the species Clostridium sartagoformum is added to the biomass as a component of at least one immobilized culture of microorganisms.

43. The process as claimed in claim 39, wherein close to a time of the addition of the microorganism of the species Clostridium sartagoformum, an additional biomass is added to the fermentation reactor, whereby a volume loading in the fermentation reactor is continuously increased by continuous addition of biomass.

44. The process as claimed in claim 39, wherein the the generation of biogas from biomass is performed at a volume loading of ≧0.5 kg oDS/m³d, of ≧4.0 kg oDS/m³d, or of ≧8.0 kg oDS/m³d.

45. The process as claimed in claim 39, wherein the microorganism of the species Clostridium sartagoformum is added to the fermentation substrate in a quantity such that, after addition, a content of the microorganism of the species Clostridium sartagoformum makes up between 10⁻⁴% and 10% of the total number of microorganisms present in the fermentation substrate.

46. The process as claimed in claim 45, wherein the microorganism of the species Clostridium sartagoformum is added to the fermentation substrate in a quantity such that, after addition, the content of the microorganism of the species Clostridium sartagoformum makes up between 10⁻³% and 1% of the total number of microorganisms present in the fermentation substrate.

47. A strain of the microorganism Clostridium sartagoformum SBG1 which is deposited at the DSMZ under the No. 19861.

48. A microorganism with a nucleic acid which has a nucleotide sequence characterized in that the nucleotide sequence contains a sequence region which has more than 99.2% sequence identity with the nucleotide sequence SEQ ID No. 1.

49. A culture of microorganisms suitable for use in a process for a fermentative generation of biogas from biomass, wherein, in the culture of micro-organisms, a microorganism Clostridium sartagoformum as claimed in claim 47 is present, and wherein the microorganism Clostridium sartagoformum makes up at least 1% of the total number of microorganisms present in the culture.

50. The culture of microorganisms as claimed in claim 49, wherein the microorganism Clostridium sartagoformum makes up at least 10% of the total number of microorganisms present in the culture.

51. The culture of microorganisms as claimed in claim 49, wherein the microorganism Clostridium sartagoformum makes up at least 50% of the total number of microorganisms present in the culture.

52. The culture of microorganisms as claimed in claim 51, wherein it is a pure culture of the microorganism Clostridium sartagoformum.

53. The culture of microorganisms as claimed in claim 49, wherein the culture of microorganisms is an immobilized culture of microorganisms.

54. A culture of microorganisms suitable for use in a process for a fermentative generation of biogas from biomass, wherein, in the culture of micro-organisms, a microorganism Clostridium sartagoformum as claimed in claim 48 is present, and wherein the microorganism Clostridium sartagoformum makes up at least 1% of the total number of microorganisms present in the culture.

55. The culture of microorganisms as claimed in claim 54, wherein the microorganism Clostridium sartagoformum makes up at least 10% of the total number of microorganisms present in the culture.

56. The culture of microorganisms as claimed in claim 55, wherein the microorganism Clostridium sartagoformum makes up at least 50% of the total number of microorganisms present in the culture.

57. The culture of microorganisms as claimed in claim 56, wherein it is a pure culture of the microorganism Clostridium sartagoformum.

58. The culture of microorganisms as claimed in claim 54, wherein the culture of microorganisms is an immobilized culture of microorganisms.


SEQUENCE LISTING (2 sequences)

SEQ ID NO: 1 - RNA, 948 nt, Clostridium Sartagoformum SBG1
cggcgugccuaccaugcaagucgagcgaggagauucccuucggggaugaaccuagcggcggacgggugaguaacacgugggcaaccugccuuauagaggggaauagccuuccgaaaggaagauuaauaccgcauaacauuacauuuucgcaugaagaaguaauuaaaggagcaauccgcuauaagaugggcccgcggcgcauuagcuaguuggugagguaacggcucaccaaggcgacgaugcguagccgaccugagagggugaucggccacauugggacugagacacggcccagacuccuacgggaggcagcaguggggaauauugcacaaugggggaaacccugaugcagcaacgccgcgugagugaugaaggucuucggaucguaaagcucugucuucagggacgauaaugacgguaccugaggaggaagccacggcuaacuacgugccagcagccgcgguaauacguagguggcgagcguuguccggauuuacugggcguaaagggagcguaggcggauuuuuaagugagaugugaaauacccgggcucaacuugggugcugcauuucaaacuggaagucuagagugcaggagaggagaguggaauuccuaguguagcggugaaaugcguagagauuaggaagaacaccaguggcgaaggcgacucucuggacuguaacugacgcugaggcucgaaagcguggggagcaaacaggauuagauacccugguaguccacgccguagacgaugaauacuagguguagggguugucaugaccucugugccgccgcagacgcauuaaguauuccgccuggggaguacggucgcaagauuaaaacucaaaggaauugacggggcccgcaccagcagcggagcaugugguuuaauucgaagcaccgcgagaaccuuaccuagacuugacaucuccugaauuacucuuaauc

SEQ ID NO: 2 - DNA, 1441 bp, Artificial Sequence
  Non-cultured Bacterial Clone aaa62b07
gacgaacgctggcggcgtgcctaacacatgcaagtcgagcgaggagattcccttcggggatgaacctagcggcggacgggtgagtaacacgtgggcaacctgccttatagaggggaatagccttccgaaaggaagattaataccgcataacattacattttcgcatgaagaagtaattaaaggagtaatccgctataagatgggcccgcggcgcattagctagttggtgaggtaacggctcaccaaggcgacgatgcgtagccgacctgagagggtgatcggccacattgggactgagacacggcccagactcctacgggaggcagcagtggggaatattgcacaatgggggaaaccctgatgcagcaacgccgcgtgagtgatgaaggtcttcggatcgtaaagctctgtcttcagggacgataatgacggtacctgaggaggaagccacggctaactacgtgccagcagccgcggtaatacgtaggtggcgagcgttgtccggatttactgggcgtaaagggagcgtaggcggatttttaagtgagatgtgaaatacccgggctcaacttgggtgctgcatttcaaactggaagtctagagtgcaggagaggagagtggaattcctagtgtagcggtgaaatgcgtagagattaggaagaacaccagtggcgaaggcgactctctggactgtaactgacgctgaggctcgaaagcgtggggagcaaacaggattagataccctggtagtccacgccgtagacgatgaatactaggtgtaggggttgtcatgacctctgtgccgccgcaaacgcattaagtattccgcctggggagtacggtcgcaagattaaaactcaaaggaattgacgggggcccgcacaagcagcggagcatgtggtttaattcgaagcaacgcgaagaaccttacctagacttgacatctcctgaattactcttaatcgaggaagtcccttcggagacaggaagacaggtggtgcatggttgtcgtcagctcgtgtcgtgagatgttgggttaagtcccgcaacgagcgcaacccttattgttagttgctaccattaagttgagcactctagcaagactgcccgggttaaccgggaggaaggtggggatgacgtcaaatcatcatgccccttatgtctagggctacacacgtgctacaatggcaagtacaaaaagatgcaataccgcaaggtggagctaaacttaaaaacttgtctcagttcggattgtaggctgaaactcgcctacatgaagctggagttgctagtaatcgcgaatcagaatgtcgcggtgaatacgttcccgggccttgtacacaccgcccgtcacaccatgagagttggcaatacccaaagttcgtgagctaacccgtaagggaggcagcgacctaaggtagggtcagcgattggggtgaag